(19) United States
(12) Patent Application Publication
(10) Pub. No.: US 2021/0007613 A1
(43) Pub. Date: Jan. 14, 2021

(54) SIGNAL PROCESSING DEVICE, SIGNAL PROCESSING METHOD, PROGRAM, AND MEASUREMENT DEVICE

(71) Applicants: Sony Semiconductor Solutions Corporation, Kanagawa (JP); Sony Corporation, Tokyo (JP)

(72) Inventors: Yuki Yagishita, Kanagawa (JP); Atsushi Ito, Kanagawa (JP)

(21) Appl. No.: 17/040,195

(22) PCT Filed: Mar. 18, 2019
(86) PCT No.: PCT/JP2019/011065

§ 371 Date: Sep. 22, 2020

(30) Foreign Application Priority Data
Mar. 29, 2018 (JP) 2018-063754

(51) Int. Cl. A61B 5/0285; A61B 5/026; A61B 5/00

(52) U.S. Cl. CPC A61B 5/0285; A61B 5/7225; A61B 5/0261

(57) ABSTRACT

The present technology relates to a signal processing device, a signal processing method, a program, and a measurement device which are capable of saving electric power while reducing a cost.

The signal processing device mixes a periodical periodic signal with a reflection signal corresponding to reflection light reflected at a subject and filters, by a low pass filter (LPF), a mixed signal acquired by mixing the reflection signal with the periodic signal. The present technology is applicable to, for example, a measurement device that non-invasively measures a blood flow velocity under the skin by irradiating a human body with light and receiving reflection light reflected at the human body.

### TECHNICAL FIELD

The present technology relates to a signal processing device, a signal processing method, a program, and a measurement device, and particularly relates to a signal processing device, a signal processing method, a program, and a measurement device which are capable of saving electric power while reducing a cost.

### BACKGROUND ART

There is a technology called a laser doppler flowmetry (LDF) method in which a blood flow velocity under the skin is non-invasively measured by irradiating the skin of a human with coherent light and analyzing backscattered light of the light, and a measurement device using the LDF method (laser doppler blood flow meter) is provided.

For example, Patent Document 1 discloses a technology in which a subject is irradiated with light, received light intensity of scattered light scattered at the subject is sampled, power only at a specific frequency is calculated from the sampled received light intensity, and a pulse waveform or a pulse rate is obtained on the basis of temporal fluctuation of the calculated power.

### CITATION LIST

### Patent Document

Patent Document 1: Japanese Patent Application Laid-Open No. 2012-005597

### SUMMARY OF THE INVENTION

### Problems to be Solved by the Invention

By the way, in a general measurement device using an LDF method, fast Fourier transform (FFT) is performed, and therefore, a calculation amount is large and it is necessary to perform high-speed digital arithmetic operation to achieve operation in real time. Therefore, the general measurement device using the LDF method includes an expensive large scale integration (LSI) such as a digital signal processor (DSP) and the like, and a cost is increased as a result thereof.

Furthermore, in the general measurement device using the LDF method, electric power can be hardly saved because it is necessary to perform the high-speed digital arithmetic operation.

The present technology is made in view of such situations and directed to achieving electric power saving while reducing a cost.

### Solutions to Problems

The signal processing device or the program of the present technology includes a signal processing device or a program that causes a computer to function as the signal processing device, in which the signal processing device includes: a mixing unit that mixes a periodical periodic signal with a reflection signal corresponding to reflection light reflected at a subject; and a low pass filter (LPF) that filters a mixed signal acquired by mixing the periodic signal with the reflection signal.

A signal processing method of the present technology includes a signal processing method including: mixing a periodical periodic signal with a reflection signal corresponding to reflection light reflected at a subject; and filtering, by a low pass filter (LPF), a mixed signal acquired by mixing the reflection signal with the periodic signal.

In the signal processing device, the signal processing method, and the program of the present technology, the reflection signal corresponding to the reflection light reflected at the subject is mixed with the periodical periodic signal, and the mixed signal acquired by mixing the reflection signal with the periodic signal is filtered by the low pass filter (LPF).

A measurement device of the present technology includes a measurement device including: a light-emitting portion that irradiates a subject with light; a light-receiving portion that receives reflection light of the light reflected at the subject and outputs a reflection signal corresponding to the reflection light; a mixing unit that mixes the reflection signal with a periodical periodic signal; a low pass filter (LPF) that filters a mixed signal acquired by mixing the reflection signal with the periodic signal; and a multiplication unit that multiplies power of an output signal of the LPF by an angular frequency of the periodic signal.

In the measurement device of the present technology, the subject is irradiated with the light, and the reflection light of the light reflected at the subject is received. Furthermore, the reflection signal corresponding to the reflection light is mixed with the periodical periodic signal, and the mixed signal acquired by mixing the reflection signal with the periodic signal is filtered by the low pass filter (LPF). Then, the power of the output signal of the LPF is multiplied by the angular frequency of the periodic signal.

### Effects of the Invention

According to the present technology, electric power can be saved while reducing a cost.

Note that the effect recited herein is not necessarily limited and may include any of effects recited in the present disclosure.

### BRIEF DESCRIPTION OF DRAWINGS

FIG. 1 is a block diagram illustrating an exemplary configuration of an embodiment of a measurement device to which the present technology is applied.

FIG. 2 is a block diagram illustrating a first exemplary configuration of an extraction unit 115.

FIG. 3 is a diagram to describe processing performed in the extraction unit 115.

FIG. 4 is a block diagram illustrating a first exemplary configuration of an arithmetic operation unit 116.

FIG. 5 is a flowchart to describe exemplary processing performed in a signal processing unit 114.

FIG. 6 is a diagram illustrating an exemplary blood flow velocity measured by a mixing method and an exemplary blood flow velocity measured by an FFT method.

FIG. 7 is a block diagram illustrating a second exemplary configuration of the extraction unit 115 and the arithmetic operation unit 116.

FIG. 8 is a block diagram illustrating a third exemplary configuration of the extraction unit 115 and the arithmetic operation unit 116.

FIG. 9 is a diagram to describe exemplary sweep of an angular frequency ωₗₒ of a periodic signal in a local oscillator 140.

FIG. 10 is a block diagram illustrating a fourth exemplary configuration of the extraction unit 115 and the arithmetic operation unit 116.

FIG. 11 is a block diagram illustrating an exemplary configuration of an embodiment of a computer to which the present technology is applied.

### MODE FOR CARRYING OUT THE INVENTION

<Measurement Principle of General Measurement Device Using LDF Method>

First, a measurement principle of a general measurement device using an LDF method will be briefly described below.

When human skin is irradiated with light of an appropriate wavelength from the outside, most of the light penetrates under the skin and is scattered at a cell membrane and various organelles. The light scattered at the cell membrane and the various organelles is again effused from the skin as backscattered light. Among such backscattered light, a light wavelength is changed due to Doppler shift in backscattered light scattered at a moving object existing under the skin, for example, at a red blood cell.

If possible to directly observe such a light wavelength change, a blood flow velocity (velocity of a tissue moving inside a human body) can be measured. However, it is actually difficult to directly observe the light wavelength change of the backscattered light caused by the Doppler shift because a light oscillation frequency (frequency) is extremely high like several hundred terahertz (THz).

By the way, the Doppler shift does not occur in backscattered light at an immobile cell (non-moving object) existing under the skin. Therefore, in a case of irradiating the human skin with coherent light, the backscattered light of the irradiation light scattered at a moving cell (moving object) interferes with the backscattered light scattered at an immobile cell (non-moving object), and optical beat including beat of the light is generated as a result thereof.

Since an oscillation frequency of this optical beat reaches to about 10 kHz, observation can be performed with an ordinary simple measurement device. A moving velocity of a moving object existing under the skin, for example, a moving velocity of a red blood cell can be obtained as a blood flow velocity by measuring a beat signal that is an electric signal of this optical beat. As described above, the method of obtaining the blood flow velocity and the like from the beat signal is the LDF method.

The general measurement device using the LDF method converts a beat signal from an analog signal to a digital signal by an analog digital converter (ADC), and the digital data acquired as a result thereof is accumulated for a predetermined time period, and then a (relative) blood flow velocity (change in a blood flow velocity) is obtained in accordance with Expression (1) below.

$$\begin{matrix}[ Expression 1 ] & \\ BLOOD FLOW VELOCITY = \frac{\int ω P ( ω ) d ω}{\int P ( ω ) d ω} & ( 1 )\end{matrix}$$

Here, in Expression (1), ω represents an angular frequency of the beat signal, and P(ω) represents power spectrum density of the beat signal.

To operate the general measurement device using the LDF method in real time, it is necessary to accumulate, for the predetermined time period, digital data acquired by performing AD conversion for a beat signal, perform arithmetic operation for the power spectrum density P(co) of the beat signal by using the digital data, and then perform arithmetic operation of integration and division in Expression (1) at a high speed.

As a specific example of measuring the blood flow velocity, for example, in a case of sampling a beat signal at 50 kHz and calculating the power spectrum density from 1024-point digital data, a time period required to sample the 1024-point digital data is about 20 ms (≈1024 points/50 kHz). Accordingly, to operate the general measurement device using the LDF method in real time, it is necessary to complete, within 20 ms or less, the arithmetic operation of Expression (1), that is, the arithmetic operation for the power spectrum density P(co) of the beat signal, integration, division, and the like in Expression (1), and in a case of exceeding 20 ms, the digital data is defected.

Here, ∫ωP(ω)dω in a numerator of Expression (1) represents an average velocity of a blood flow, and furthermore, the division by ∫P(ω)dω is performed in Expression (1) to cancel influence of a change in the power of the light emitted to a human body, and the like. In Expression (1), not an absolute blood flow velocity but a relative blood flow velocity is obtained, and therefore, the blood flow velocity obtained by Expression (1) is used to observe the change in the blood flow velocity.

As described above, the arithmetic operation in Expression (1) is required to be performed at the high speed in the general measurement device using the LDF method. Accordingly, the general measurement device using the LDF method often includes a DSP in order to perform the arithmetic operation in Expression (1) at the high speed. However, since an expensive LSI is used for the DSP, a cost for the general measurement device using the LDF method is increased, and moreover, electric power consumption is increased because the arithmetic operation in Expression (1) is required to be performed at the high speed.

As a measure against such increase in the cost and electric power consumption of the general measurement device using the LDF method, Patent Document 1 discloses a technology in which a calculation amount is reduced by calculating only intensity of a beat signal relative to a specific angular frequency without performing the arithmetic operation for the power spectrum density, and moreover, a cost is reduced by making it possible to constitute a measurement device without using a DSP.

By the way, the angular frequency co of the beat signal is proportional to a moving velocity v of a moving object existing under the skin, for example, a particle such as a red blood cell, and therefore, it is known that the angular frequency ω of the beat signal and a moving velocity v of a particle can be expressed by a relational expression shown in Expression (2) below.

[Expression 2]

ω=|v∥kᵢ−kₛ| (2)

However, note that, in Expression (2), v represents a velocity vector of the particle (one object such as a red blood cell), and kᵢ represents an incident light vector indicating intensity and a direction (in which a wavefront advances) of incident light. kₛ represents a scattered light vector indicating intensity and a direction of the scattered light.

According to Expression (2), the particle velocity v and the angular frequency ω of the beat signal have a one-to-one proportional relation. Accordingly, one angular frequency ω of a beat signal corresponds to the velocity v of one particle.

The measuring method for the blood flow velocity disclosed in Patent Document 1 is a measuring method focusing only on the specific angular frequency, and therefore, the measuring method is equivalent to measuring an amount of particles having a specific velocity corresponding to the specific angular frequency.

Since the general measurement device using the LDF method measures the blood flow velocity from all of angular frequencies (an average moving velocity of a plurality of moving particles), it can be said that the measuring method focusing only on the specific angular frequency as disclosed in Patent Document 1 performs the measurement substantially different from the measurement in the general measurement device using the LDF method, that is, the measurement device that measures a blood flow velocity by performing the arithmetic operation in Expression (1).

Furthermore, in the technology disclosed in Patent Document 1, there is a possibility that the DSP becomes unnecessary because the power spectrum density is not calculated, however, the calculation amount is large because 4096-point addition and subtraction are performed (recited in paragraphs [0041] to [0042] and the like in Patent Document 1). Therefore, even though the DSP can be made unnecessary, it is difficult to achieve significant electric power saving.

In addition, there is a high possibility that a high-speed ADC is required, which causes cost increase in the technology disclosed in Patent Document 1.

<Embodiment of Measurement Device to which Present Technology is Applied>

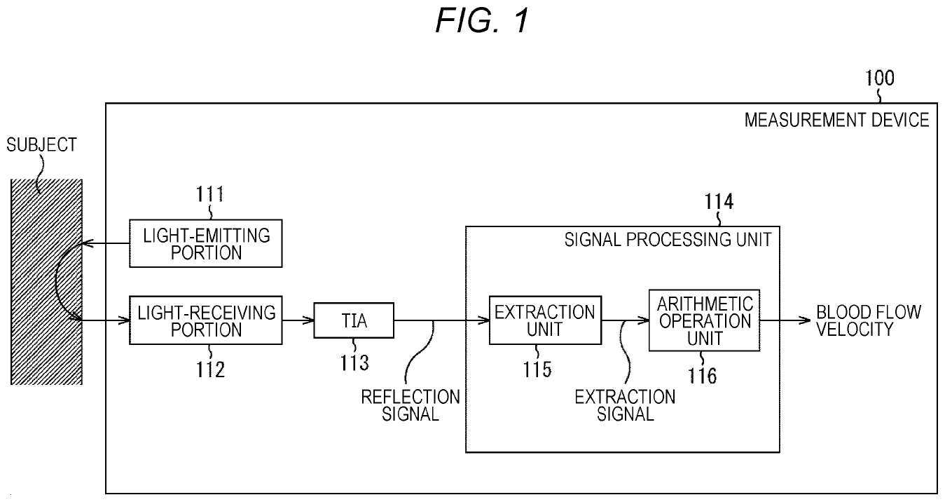

FIG. 1 is a block diagram illustrating an exemplary configuration of an embodiment of a measurement device to which the present technology is applied.

The measurement device 100 illustrated in FIG. 1 includes a light-emitting portion 111, a light-receiving portion 112, a trans impedance amplifier (TIA) 113, and a signal processing unit 114.

The light-emitting portion 111 is a light source that emits light that is at least partially coherent, and irradiates a subject, for example, a human body or the like with the light. As the light-emitting portion 111, it is possible to use, for example, a light source that emits light in a single mode, such as a laser diode (LD) of a distributed feedback (DFB) type, a vertical cavity surface emitting laser (VCSEL), or the like.

The light-receiving portion 112 includes, for example, a photo diode (PD) using a material such as silicon (Si). The light-receiving portion 112 receives reflection light (backscattered light) of the light that is emitted from the light-emitting portion 111 (hereinafter referred to as irradiation light) and scattered as reflection at a subject, for example, a tissue existing under the skin of the human body, and the light-receiving portion 112 photoelectrically converts the received reflection light.

A part of the irradiation light emitted from the light-emitting portion 111 is scattered at a particle moving inside the human body, for example, at a red blood cell or the like, and the Doppler shift occurs. The reflection light from the human body as the subject includes: reflection light in which the Doppler shift has occurred; and reflection light in which the Doppler shift has not occurred, and because of interference between the reflection light in which the Doppler shift has occurred and the reflection light the Doppler shift has not occurred, the optical beat that is random oscillation is observed.

The light-receiving portion 112 photoelectrically converts the reflection light from the human body as described above. Then, the light-receiving portion 112 supplies, to the TIA 113, a reflection signal (beat signal) that is acquired by the photoelectric conversion and corresponds to the reflection light.

The TIA 113 applies, to the reflection signal supplied from the light-receiving portion 112, current-voltage conversion that converts current into voltage, amplifies the voltage as the reflection signal to an extent at which electric processing can be performed, and supplies the amplified signal to the signal processing unit 114.

The signal processing unit 114 has an extraction unit 115 and an arithmetic operation unit 116.

The extraction unit 115 mixes (multiplies) a periodical periodic signal with (by) the reflection signal supplied from the TIA 113 to the signal processing unit 114, extracts a frequency component of a predetermined (angular) frequency band from a mixed signal acquired by filtering, by a low pass filter (LPF), the mixed signal acquired by mixing the reflection signal with the periodic signal, and supplies the extracted frequency component as an extraction signal to the arithmetic operation unit 116.

The arithmetic operation unit 116 obtains power of the extraction signal supplied from the extraction unit 115 and multiplies the power of the extraction signal by an angular frequency of the periodic signal. Then, the arithmetic operation unit 116 obtains a blood flow velocity from a multiplication value acquired as a result of multiplying the power of the extraction signal by the angular frequency of the periodic signal, and outputs the blood flow velocity.

<First Exemplary Configuration of Extraction Unit 115 and Arithmetic Operation Unit 116>

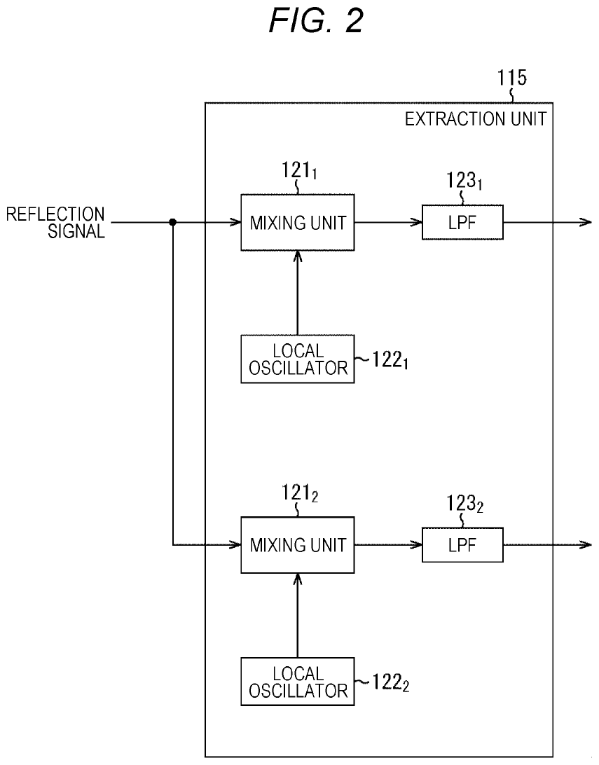

FIG. 2 is a block diagram illustrating a first exemplary configuration of the extraction unit 115 in FIG. 1.

The extraction unit 115 illustrated in FIG. 2 includes mixing units 121₁ and 121₂, local oscillators 122₁ and 122₂, and LPFs 123₁ and 123₂.

The mixing unit 121₁ mixes a reflection signal supplied from the TIA 113 with a periodic signal supplied from the local oscillator 122₁ and supplies, to the LPF 123₁, a mixed signal acquired by mixing the reflection signal with the periodic signal.

The local oscillator (LO) 122₁ generates, for example, by oscillation, a periodical periodic signal having a sine wave, a rectangular wave, or the like of a predetermined angular frequency and supplies the periodic signal to the mixing unit 121₁.

The LPF 123₁ filters the mixed signal supplied from the mixing unit 121₁ and supplies, as an extraction signal to the arithmetic operation unit 116, a frequency component of a low angular frequency of the mixed signal acquired by the filtering.

The mixing unit 121₂, the local oscillator 122₂, and the LPF 123₂ have configurations similar to those of the mixing unit 121₁, the local oscillator 122₁, and the LPF 123₁, respectively, and therefore, a description thereof will be omitted. However, note that the local oscillator 122₂ generates a periodic signal having an angular frequency different from that in the local oscillator 122₁.

Here, a reflection signal supplied from the TIA 113 to the mixing unit 121ₙ of the extraction unit 115 is represented as A sin(ωₜᵢₐ+φ), and a periodic signal generated by a local oscillator 122ₙ is represented as sin (ωₗₒt). In FIG. 2, n=1, 2.

ωₜᵢₐ represents an angular frequency of the reflection signal, and ωₗₒ represents an angular frequency of the periodic signal. φ represents a phase (deviation) of the reflection signal A sin(ωₜᵢₐ+φ) with respect to the periodic signal sin (ωₗₒt).

In the mixing unit 121ₙ, the reflection signal A sin(ωₜᵢₐ+φ) and the periodic signal sin (ωₗₒt) are mixed, that is, multiplied, and the mixed signal A sin(ωₜᵢₐ+φ)×sin (ωₗₒt) shown in Expression (3) below is acquired.

$$\begin{matrix} [ Expression 3 ] & \\ Asin ( {ω}_{tia} t + Φ ) \times sin ( {ω}_{lo} t ) = - \frac{A}{2} [ cos \{ ( {ω}_{tia} + {ω}_{lo} ) t + Φ \} - cos \{ ( {ω}_{tia} - {ω}_{lo} ) t + Φ \} ] & ( 3 )\end{matrix}$$

The mixed signal A sin(ωₜᵢₐ+φ)×sin (ωₗₒt) shown in Expression (3) is supplied to an LPF 123ₙ from the mixing unit 121ₙ, and the mixed signal A sin(ωₜᵢₐ+φ)×sin (ωₗₒt) shown in Expression (3) is filtered at the LPF 123ₙ. That is, in the LPF 123ₙ, a frequency component−A/2 cos{((ωₜᵢₐ+ωₗₒ)t+φ} of a high angular frequency (ωₜᵢₐ+ωₗₒ) is removed from the mixed signal A sin(ωₜᵢₐ+φ)×sin (ωₗₒt)=−A/2 [cos{(ωₜᵢₐ+ωₗₒ) t+}−cos{(ωₜᵢₐ−ωₗₒ)t+φ}], and only a frequency component A/2 cos {(ωₜᵢₐ−ωₗₒ) t+φ} of a low angular frequency (ωₜᵢₐ−ωₗₒ) is extracted and output as the extraction signal. Accordingly, the extraction signal is expressed as Expression (4) below.

$$\begin{matrix}[ Expression 4 ] & \\ \frac{A}{2} cos \{ ( {ω}_{tia} - {ω}_{lo} ) t + Φ \} & ( 4 )\end{matrix}$$

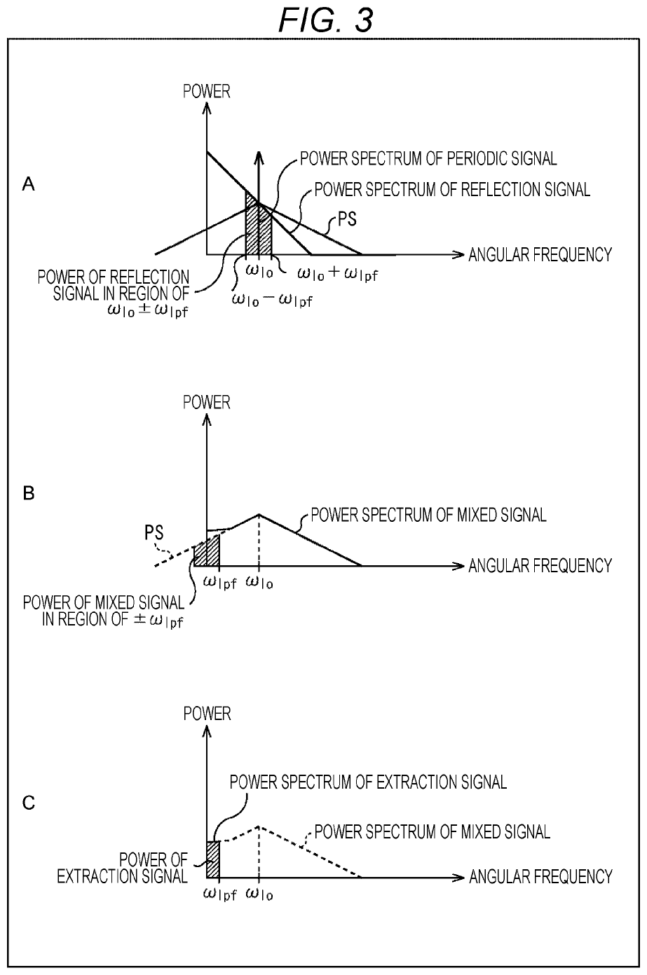

FIG. 3 is a diagram to describe processing performed in the extraction unit 115.

In FIG. 3, a vertical axis represents signal power and a horizontal axis represents an angular frequency.

A of FIG. 3 illustrates a power spectrum of a reflection signal and a power spectrum of a periodic signal having an angular frequency ωₗₒ.

Additionally, A of FIG. 3 illustrates a power spectrum PS combining: a power spectrum in which an amplitude of power of the power spectrum of the reflection signal is halved and the power spectrum is shifted to a positive side of the angular frequency by the angular frequency ωₗₒ; and a power spectrum in which such a power spectrum is folded back at the angular frequency ωₗₒ.

B of FIG. 3 illustrates a power spectrum of a mixed signal acquired by mixing, in the mixing unit 121″ the reflection signal with the periodic signal in A of FIG. 3.

The power spectrum of the mixed signal is to be a power spectrum acquired by folding back, at 0 Hz, a frequency component on a negative side of the angular frequency of the power spectrum PS in A of FIG. 3 and adding this folded frequency component to the frequency component on the positive side. In B of FIG. 3, the power spectrum of the mixed signal output from the mixing unit 121ₙ is indicated by a solid line.

C of FIG. 3 illustrates a power spectrum of an extraction signal that is the output signal of the LPF 123ₙ. In the LPF 123ₙ, the mixed signal in B of FIG. 3 is filtered, and a signal which is included in the mixed signal and corresponds to a signal of the power spectrum indicated by a solid line in C of FIG. 3, that is, a signal of a frequency component of a low angular frequency equal to or less than a cutoff angular frequency ω_{lpf} of the LPF 123ₙ is output as the extraction signal.

In C of FIG. 3, power of the extraction signal is indicated by a shaded region and corresponds to power in a shaded region in each of A and B of FIG. 3. The extraction signal corresponds to a signal acquired by extracting a signal in an (angular) frequency band from an angular frequency ωₗₒ−ω_{lpf} of the reflection signal (A in FIG. 3) to an angular frequency ωₗₒ+ω_{lpf} (A in FIG. 3).

Hereinafter, the power of the extraction signal output from the LPF 123ₙ is represented as P(ωₗₒ) relative to the angular frequency ωₗₒ. The power P(ωₗₒ) of the extraction signal corresponds to P(ω) of ∫ωP(ω)dω in the numerator of Expression (1) that obtains the blood flow velocity.

Here, the cutoff angular frequency ω_{lpf} can be determined by, for example, simulation and the like so as to improve (measurement) accuracy of the blood flow velocity obtained by using P(ωₗₒ) as described later.

∫ωP(ω)dω in the numerator of Expression (1) that obtains the blood flow velocity can be transformed into Expression (5) below.

$$\begin{matrix} [ Expression 5 ] & \\ \int ω P ( ω ) d ω = \sum ω P ( ω ) = {ω}_{lo 1} P ( {ω}_{lo 1} ) + {ω}_{lo 2} P ( {ω}_{lo 2} ) + {ω}_{lo 3} P ( {ω}_{lo 3} ) + … + {ω}_{loN} P ( {ω}_{loN} ) & ( 5 )\end{matrix}$$

Note that ω_{lo #n} in Expression (5) represents an angular frequency of a periodic signal, and P(ω_{lo #n}) represents power of an extraction signal acquired by mixing a reflection signal relative to the angular frequency ω_{lo #n}, that is, mixing the reflection signal with a periodic signal of the angular frequency ω_{lo #n}, and filtering the mixed signal.

Furthermore, N is an integer of 1 or more, and angular frequencies ωₗₒ₁ to ω_{loN} represent different angular frequencies, respectively. Moreover, the angular frequencies ωₗₒ₁ to ω_{loN} are assumed to take values in a range of the integration represented in Expression (1) (for example, several KHz to several tens KHz), and an interval between the values, that is, an interval between the values of the angular frequency ω_{lo #n} and an angular frequency ω_{lo #n+1} may be a constant interval or may not be a constant interval. Additionally, the angular frequencies ωₗₒ₁ to ω_{loN} may be values arrayed in ascending order or descending order, or may be values not arrayed in the ascending order or the descending order.

Expression (5) that obtains a blood flow velocity can be approximated as shown in Expression (6) below, for example.

[Expression 6]

ωₗₒ₁P(ωₗₒ₁)+ωₗₒ₁P(ωₗₒ₂)+ωₗₒ₃P(ωₗₒ₃)+ . . . +ω_{loN}(ω_{loN})≈ωₗₒ₁P(ωₗₒ₁)+ωₗₒ₂P(ωₗₒ₂) (6)

According to Expression (6), the blood flow velocity can be approximated by arithmetic operation of ωₗₒ₁P(ωₗₒ₁)+ωₗₒ₂P(ωₗₒ₂). That is, the blood flow velocity can be obtained from: power P(ωₗₒ₁) and power P(ωₗₒ₂) of two extraction signals and the angular frequencies ωₗₒ₁ and ωₗₒ₂ of periodic signals used to extract the two extraction signals, in which the two extraction signals are each extracted by mixing a reflection signal with a periodic signal of each of two different angular frequencies ωₗₒ₁ and ωₗₒ₂.

The two extraction signals relative to the angular frequencies ωₗₒ₁ and ωₗₒ₂ are obtained in the extraction unit 115 in FIG. 2, and the extraction signals are supplied to the arithmetic operation unit 116 in FIG. 1. The arithmetic operation unit 116 in FIG. 1 performs the arithmetic operation in accordance with Expression (6) by using the two extraction signals supplied from the extraction unit 115, and the blood flow velocity is obtained.

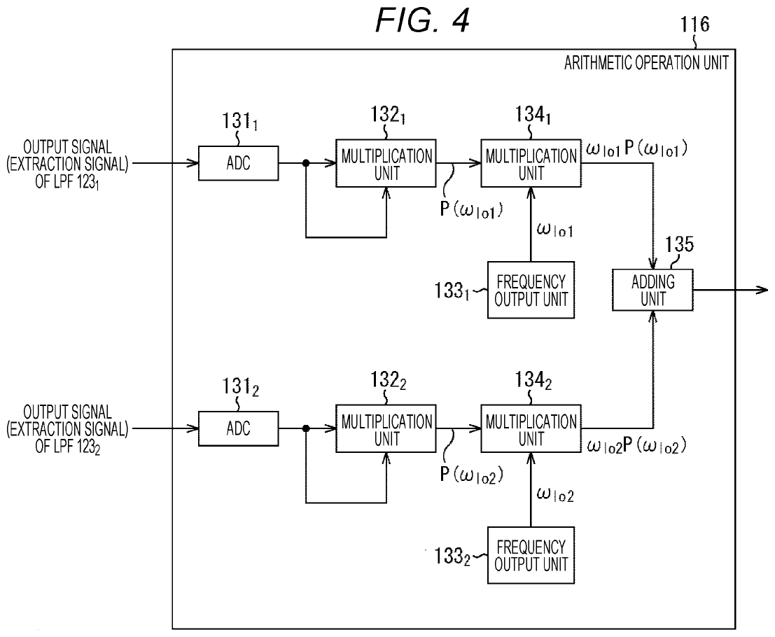

FIG. 4 is a block diagram illustrating a first exemplary configuration of an arithmetic operation unit 116 in FIG. 1.

The arithmetic operation unit 116 illustrated in FIG. 4 includes ADCs 131₁ and 131₂, multiplication units 132₁ and 132₂, frequency output units 133₁ and 133₂, multiplication units 134₁ and 134₂, and an adding unit 135.

The ADC 131ₙ (n=1, 2 in FIG. 4) performs AD conversion for an (analog) extraction signal relative to the angular frequency ω_{lo #n} supplied from the LPF 123ₙ in the extraction unit 115 (FIG. 2) and supplies, to the multiplication unit 132ₙ, a digital extraction signal acquired by the AD conversion.

The multiplication unit 132ₙ obtains power P(ω_{lo #n}) of the extraction signal by squaring the digital extraction signal supplied from the ADC 131ₙ, and supplies the power P(ω_{lo #n}) to the multiplication unit 134ₙ.

Here, the multiplication unit 132ₙ can obtain the power P(ω_{lo #n}) of the extraction signal not only by squaring each sample value of the extraction signal but also by squaring, in every certain time section, an average value of sample values of extraction signals during the time section. In this case, as for each sample point during the certain time section, a value obtained by squaring the average value of the sample values of the extraction signals during the time section is obtained as the power P(ω_{lo #n}) of the extraction signal.

The frequency output unit 133ₙ supplies, to the multiplication unit 134ₙ, the angular frequency ω_{lo #n} of the periodic signal used to obtain the extraction signal output from the LPF 123ₙ, that is, the angular frequency ω_{lo #n} of the periodic signal generated at the local oscillator 122ₙ.

The multiplication unit 134ₙ multiplies the power P(ω_{lo #n}) of the extraction signal supplied from the multiplication unit 132ₙ by the angular frequency ω_{lo #n} supplied from the frequency output unit 133ₙ and supplies, to the adding unit 135, a multiplication value ω_{lo #n}P (ω_{lo #n}) acquired as a result of the multiplication.

The adding unit 135 adds a multiplication value ωₗₒ₁P(ωₗₒ₁) supplied from the multiplication unit 134₁ and a multiplication value ωₗₒ₂P(ωₗₒ₂) supplied from the multiplication unit 134₂ and outputs, as a blood flow velocity to the outside, an added value ωₗₒ₁P(ωₗₒ₁)+ωₗₒ₂P(ωₗₒ₂) acquired as a result of the addition.

Note that, in the arithmetic operation unit 116 of FIG. 4, calculation of power P(ω_{lo #n}) of an extraction signal, multiplication of the power P(ω_{lo #n}) of the extraction signal by an angular frequency ω_{lo #n}, and addition of multiplication values ω_{lo #n}P(ω_{lo #n}) acquired by the multiplication are performed for a digital signal, but the calculation of the power P(ω_{lo #n}) of the extraction signal, the multiplication of the power P(ω_{lo #n}) of the extraction signal by the angular frequency ω_{lo #n}, and addition of the multiplication values ω_{lo #n}P(ω_{lo #n}) can be performed for an analog signal.

As described above, in the case of performing the calculation of the power P(ω_{lo #n}) of the extraction signal, the multiplication of the power P(ω_{lo #n}) of the extraction signal by the angular frequency ω_{lo #n}, and the addition of the multiplication values ω_{lo #n}P(ω_{lo #n}) for an analog signal, the multiplication unit 132ₙ, the multiplication unit 134ₙ, and the adding unit 135 of the arithmetic operation unit 116 each include an analog circuit that can perform arithmetic operation for the analog signal. Furthermore, the ADC 131ₙ becomes unnecessary in the arithmetic operation unit 116.

Here, in the measurement device 100, a course of performing the processing until obtaining a multiplication value ω_{lo #n}P(ω_{lo #n}) from a reflection signal is referred to as a path. In the first exemplary configuration of the extraction unit 115 and the arithmetic operation unit 116, two paths are provided, and the blood flow velocity is obtained by adding the multiplication values ωₗₒ₁P(ωₗₒ₁) and ωₗₒ₂P(ωₗₒ₂) acquired in the two respective paths.

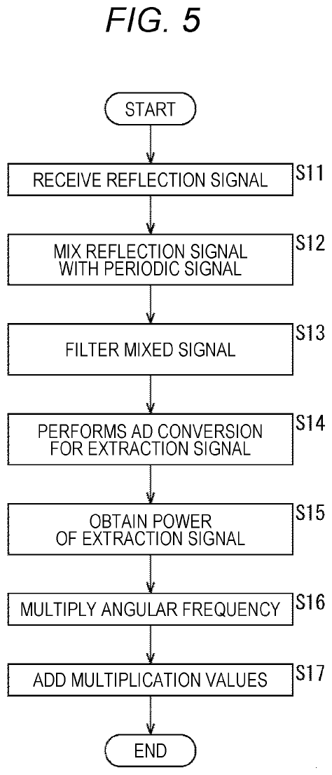

FIG. 5 is a flowchart to describe exemplary processing performed in the signal processing unit 114.

In step S11, the mixing unit 121ₙ of the signal processing unit 114 receives a reflection signal supplied from the TIA 113, and the processing proceeds to step S12.

In step S12, the mixing unit 121ₙ of the signal processing unit 114 mixes the reflection signal supplied from the TIA 113 with a periodic signal supplied from the local oscillator 122ₙ, and a mixed signal acquired from the mixing is supplied to the LPF 123ₙ, and then the processing proceeds to step S13.

In step S13, the LPF 123ₙ of the signal processing unit 114 filters the mixed signal supplied from the mixing unit 121ₙ. That is, in step S13, the LPF 123ₙ of the signal processing unit 114 extracts, from the mixed signal, a frequency component of a low angular frequency (of an (angular) frequency band equal to or lower than the cutoff angular frequency ω_{lpf}) and supplies the frequency component as an extraction signal to the ADC 131ₙ, and the processing proceeds to step S14.

In step S14, the ADC 131ₙ of the signal processing unit 114 performs AD conversion for the extraction signal supplied from the LPF 123ₙ and supplies the converted extraction signal to the multiplication unit 132ₙ, and the processing proceeds to step S15.

In step S15, the multiplication unit 132ₙ of the signal processing unit 114 obtains power P(ω_{lo #n}) of the extraction signal by squaring the extraction signal supplied from the ADC 131ₙ and supplies the power P(ω_{lo #n}) to the multiplication unit 134ₙ, and the processing proceeds to step S16.

In step S16, the multiplication unit 134ₙ of the signal processing unit 114 multiplies the power P(ω_{lo #n}) of the extraction signal supplied from the multiplication unit 132ₙ by an angular frequency ω_{lo #n} of a periodic signal supplied from the frequency output unit 133ₙ and used to obtain the extraction signal, and supplies a multiplication value ω_{lo #n}P (ω_{lo #n}) acquired by the multiplication to the adding unit 135, and the processing proceeds to step S17.

In step S17, the adding unit 135 of the signal processing unit 114 adds the multiplication values ωₗₒ₁P(ωₗₒ₁) and ωₗₒ₂P(ωₗₒ₂) respectively supplied from the multiplication units 134₁ and 134₂, and an added value ωₗₒ₁P(ωₗₒ₁)+ωₗₒ₂P(ωₗₒ₂) acquired as a result thereof is output as a blood flow velocity, and the processing ends.

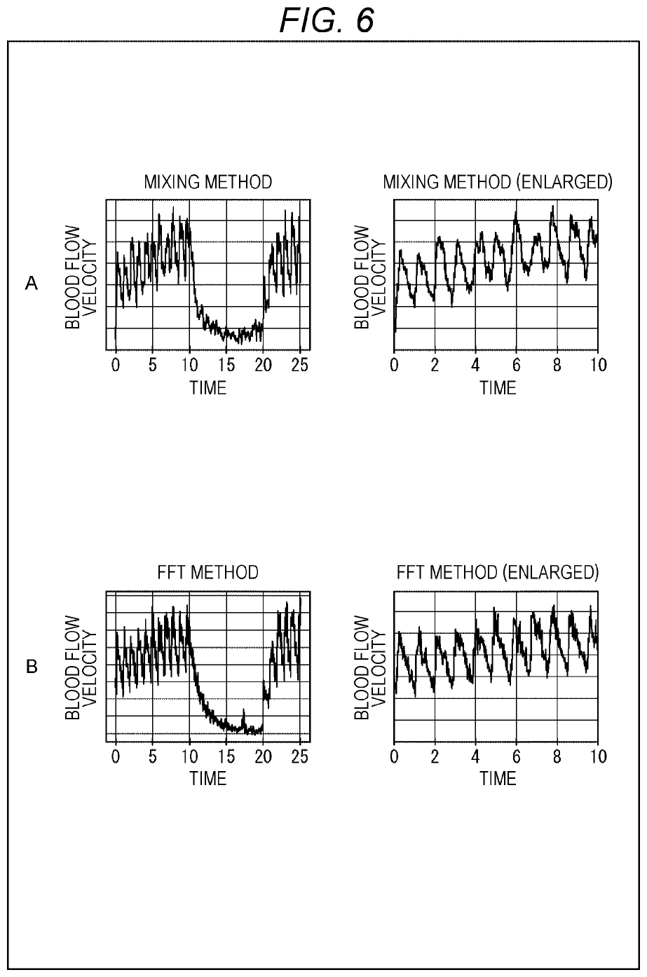

FIG. 6 is a diagram illustrating an exemplary blood flow velocity obtained by a mixing method and an exemplary blood flow velocity obtained by an FFT method.

Here, the mixing method stands for a method of mixing a reflection signal with a periodic signal and obtaining a blood flow velocity by using an extraction signal acquired by passing, through the LPF, the mixed signal acquired by the mixing, in a manner similar to the measurement device 100 illustrated in FIG. 1.

The FFT method stands for a method of performing AD conversion for a reflection signal, obtaining power P(ω) of the reflection signal by performing FFT for the reflection signal subjected to the AD conversion, and obtaining a blood flow velocity in accordance with Expression (1) by using the power P(ω) of the reflection signal, in a manner similar to the general measurement device using the LDF method.

In FIG. 6, a vertical axis represents a (relative) blood flow velocity, and a horizontal axis represents a time period (seconds).

A of FIG. 6 illustrates the blood flow velocity obtained by the mixing method and an enlarged view in which a part of the blood flow velocity is enlarged.

B of FIG. 6 illustrates the blood flow velocity obtained by the FFT method and an enlarged view in which a part of the blood flow velocity is enlarged.

Comparing the blood flow velocity obtained by the mixing method in A of FIG. 6 with the blood flow velocity obtained by the FFT method in B of FIG. 6, it can be recognized that the blood flow velocity substantially similar to that of the FFT method can be obtained by the mixing method.

That is, FIG. 6 illustrates measurement results of the blood flow velocity at a fingertip obtained by the mixing method and the FFT method in a case where a subject is in a resting state from 0 to 10 seconds, an arm is tightened from 10 to 20 seconds, and the subject is again put back to the resting state from 20 seconds and thereafter. According to FIG. 6, it can be understood that the blood flow velocity can be obtained by the mixing method with accuracy substantially similar to that in the FFT method.

Note that the blood flow velocity obtained by the mixing method in A of FIG. 6 is the blood flow velocity in the case where the extraction unit 115 and the arithmetic operation unit 116 include the two paths as illustrated in FIGS. 2 and 4.

According to the mixing method, the blood flow velocity can be obtained by using an extraction signal acquired by passing, through the LPF, a mixed signal acquired by mixing a reflection signal with a periodic signal, and therefore, it is not necessary to perform the FFT for the reflection signal. Accordingly, according to the mixing method, a calculation amount is more reduced than in the FFT method, and it is possible to easily observe a blood flow velocity in real time with little delay.

Furthermore, according to the mixing method, the calculation amount is more reduced than in the FFT method, and therefore, it is not necessary to perform high-speed digital arithmetic operation, and electric power can be saved.

Moreover, according to the mixing method, since it is not necessary to perform the high-speed digital arithmetic operation, an expensive LSI such as a DSP to perform the high-speed digital arithmetic operation becomes unnecessary, and cost reduction and miniaturization can be achieved.

In addition, the high-speed ADC is required in the technology disclosed in Patent Document 1, but the blood flow velocity can be obtained without using the high-speed ADC in the mixing method. Furthermore, in the mixing method, the arithmetic operation unit 116 can perform arithmetic operation with an analog signal, and in a case of performing the arithmetic operation with the analog signal, the arithmetic operation unit 116 can be constituted without using the ADCs 131₁ and 131₂. In a case of constituting the arithmetic operation unit 116 without using the ADCs 131₁ and 131₂, the cost can be reduced.

<Second Exemplary Configuration of Extraction Unit 115 and Arithmetic Operation Unit 116>

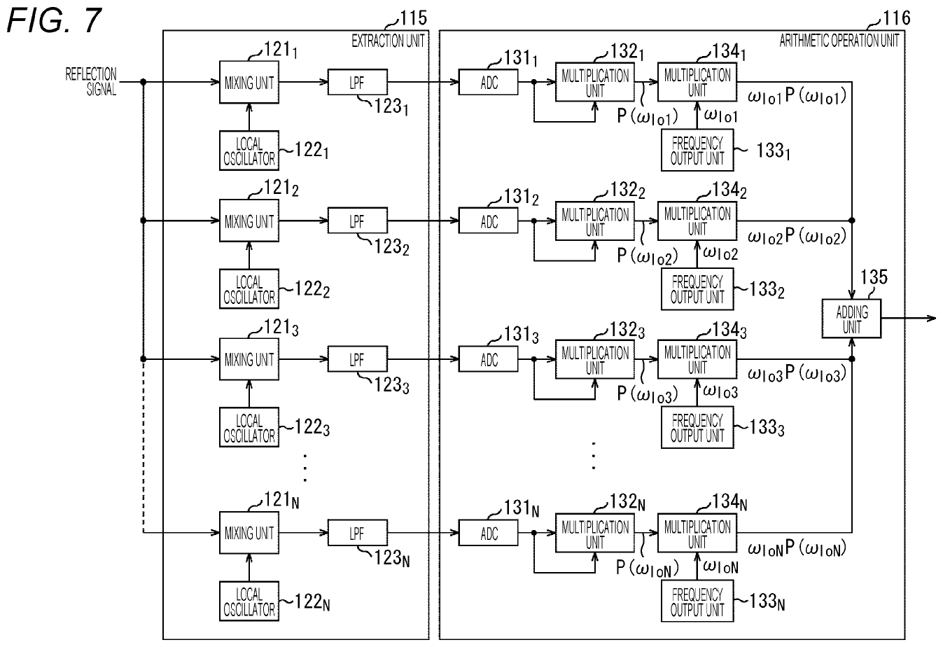

FIG. 7 is a block diagram illustrating a second exemplary configuration of the extraction unit 115 and the arithmetic operation unit 116.

Note that, in the drawing, portions corresponding to those of FIGS. 2 and 4 are denoted by the same reference signs, and a description therefor will be omitted below as appropriate.

In FIG. 7, the extraction unit 115 and the arithmetic operation unit 116 have N paths that are three or more paths.

That is, the extraction unit 115 includes mixing units 121₁ to 121_{N}, local oscillators 122₁ to 122_{N}, and LPFs 123₁ to 123_{N}. The arithmetic operation unit 116 includes ADCs 131₁ to 131_{N}, multiplication units 132₁ to 132_{N}, frequency output units 133₁ to 133_{N}, multiplication units 134₁ to 134_{N}, and an adding unit 135.

In the measurement device 100 having the configuration as described above, a reflection signal is mixed with each of N periodic signals having different angular frequencies ωₗₒ₁ to ω_{loN} in each of the mixing units 121₁ to 121_{N}, and N mixed signals acquired as a result of the mixing are supplied to the LPFs 123₁ to 123_{N} respectively.

In the LPFs 123₁ to 123_{N}, the N mixed signals supplied from the mixing units 121₁ to 121_{N} are filtered respectively, and N extraction signals acquired as a result of the filtering are supplied to the ADCs 131₁ to 131_{N} respectively.

In the ADCs 131₁ to 131_{N}, AD conversion is performed for the N extraction signals supplied from the LPFs 123₁ to 123_{N} respectively, and the N extraction signals are supplied to the multiplication units 132₁ to 132_{N} respectively.

In the multiplication units 132₁ to 132_{N}, the power P(ω_{lo #n}) of each of the N extraction signals supplied from each of the ADCs 131₁ to 131_{N} is obtained and supplied to each of the multiplication units 134₁ to 134_{N}.

In the multiplication units 134₁ to 134_{N}, respective pieces of the power P(ωₗₒ₁) to P(ω_{loN}) of the N extraction signals supplied from the multiplication units 132₁ to 132_{N} are multiplied by the respective angular frequencies ωₗₒ₁ to ω_{loN} supplied from the frequency output units 133₁ to 133_{N}, that is, the angular frequencies ωₗₒ₁ to ω_{loN} of the periodic signals used to obtain the extraction signals, and multiplication values ωₗₒ₁P(ωₗₒ₁) to ω_{loN}P(ω_{loN}) acquired as a result of the multiplication are supplied to the adding unit 135.

In the adding unit 135, the multiplication values ωₗₒ₁P(ωₗₒ₁) to ω_{loN}P(ω_{loN}) supplied from the multiplication units 134₁ to 134_{N} are added, and an added value ωₗₒ₁P(ωₗₒ₁)+ωₗₒ₂P(ωₗₒ₂)+ . . . +ω_{loN}P(ω_{loN}) acquired as a result of the addition is output as (a measurement result of) a blood flow velocity.

As obvious from the above-described Expression (5), in the mixing method, when the number N of the angular frequency ω_{lo #n} is large to some extent, the more (the measurement result of) the blood flow velocity is approximated to a result of the arithmetic operation of ∫ωP(ω)dω in the numerator of Expression (1), and therefore, the larger the number of paths is to some extent, the more improved (measurement) accuracy of the blood flow velocity can be.

Note that the number N of the angular frequencies ω_{lo #n}, that is, the number of paths can be determined by, for example, simulation and the like so as to improve the accuracy of the blood flow velocity obtained in the signal processing unit 114.

Here, it is necessary to provide the measurement device 100 with as many paths as the number N of the angular frequencies ω_{lo #n}. Accordingly, in a case of increasing the number N of angular frequencies ω_{lo #n}, a circuit scale of the measurement device 100 is increased in proportion to the number N.

Considering such a case, a description will be provided for an exemplary configuration of the extraction unit 115 and the arithmetic operation unit 116, in which the circuit scale of the measurement device 100 is not increased even though the number N of the angular frequencies ω_{lo #n} is large.

<Third Exemplary Configuration of Extraction Unit 115 and Arithmetic Operation Unit 116>

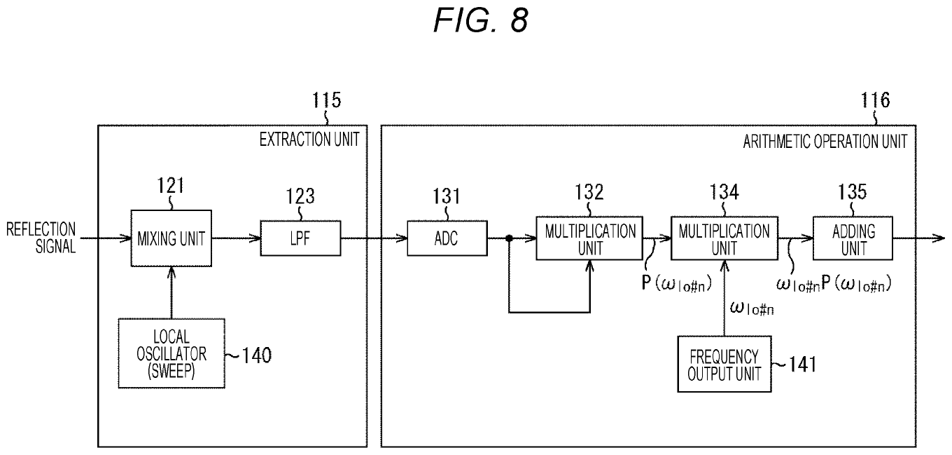

FIG. 8 is a block diagram illustrating a third exemplary configuration of the extraction unit 115 and the arithmetic operation unit 116.

In FIG. 8, the extraction unit 115 and the arithmetic operation unit 116 have one path.

That is, the extraction unit 115 includes a mixing unit 121, an LPF 123, and a local oscillator 140. The arithmetic operation unit 116 includes an ADC 131, a multiplication unit 132, a frequency output unit 141, a multiplication unit 134, and an adding unit 135.

The mixing unit 121 sequentially multiplies (mixes) a reflection signal supplied from the TIA 113 with each of a plurality of periodic signals which is sequentially supplied (in time series) from the local oscillator 140 and have different angular frequencies, and the mixing unit sequentially supplies, to the LPF 123, mixed signals relative to the plurality of angular frequencies.

The LPF 123 sequentially filters each of the mixed signals sequentially supplied from the mixing unit 121 relative to the plurality of angular frequencies, thereby sequentially extracting, from the mixed signals relative to the plurality of angular frequencies, extraction signals relative to the plurality of angular frequencies, and sequentially supplies the extraction signals to the ADC 131.

The ADC 131 sequentially performs AD conversion for the extraction signals relative to the plurality of angular frequencies supplied from the LPF 123, and sequentially supplies the converted extraction signals to the multiplication unit 132.

The multiplication unit 132 sequentially obtains pieces of power P(ω_{lo #n}) of the extraction signals sequentially supplied from the ADC 131 (n=1, 2, . . . , N) relative to the plurality of angular frequencies, and sequentially supplies the pieces of power P(ω_{lo #n}) to the multiplication unit 134.

The multiplication unit 134 sequentially multiplies the pieces of the power P(ωₗₒ₁), P(ωₗₒ₂), . . . , P(ω_{loN}) of the plurality of extraction signals supplied from the multiplication unit 132 relative to the plurality of angular frequencies sequentially by the angular frequencies ωₗₒ₁, ωₗₒ₂, . . . , ω_{loN} sequentially supplied from the frequency output unit 141, and sequentially supplies, to the adding unit 135, multiplication values ω_{lo #n}P (ω_{lo #n}) acquired as a result of the multiplication relative to the plurality of angular frequencies ω_{lo #n}.

The adding unit 135 sequentially adds the multiplication values ω_{lo #n}P(ω_{lo #n}) sequentially supplied from the multiplication unit 134 relative to the plurality of angular frequencies ω_{lo #n}, and outputs, as a blood flow velocity, an added value acquired as a result of the addition.

The local oscillator 140 sweeps (changes) an angular frequency ω_{lo #n} of a periodic signal, for example, at every predetermined time period, generates a plurality of periodic signals having the different angular frequencies ω_{lo #n}=ωₗₒ₁, ωₗₒ₂, . . . , ω_{loN}, and sequentially supplies the periodic signals to the mixing unit 121.

The frequency output unit 141 sequentially supplies, to the multiplication unit 134, the angular frequencies ω_{lo #n} of the periodic signals used to obtain the extraction signals output by the LPF 123, that is, the angular frequencies ω_{lo #n} of the periodic signals generated at the local oscillator 140.

In the extraction unit 115 and the arithmetic operation unit 116 having the configuration as described above, the processing to be performed in the mixing unit 121, the LPF 123, the ADC 131, and the adding unit 135 is performed in time series for each of the plurality of angular frequencies ω_{lo #n}=ωₗₒ₁, ωₗₒ₂, . . . , ω_{loN}.

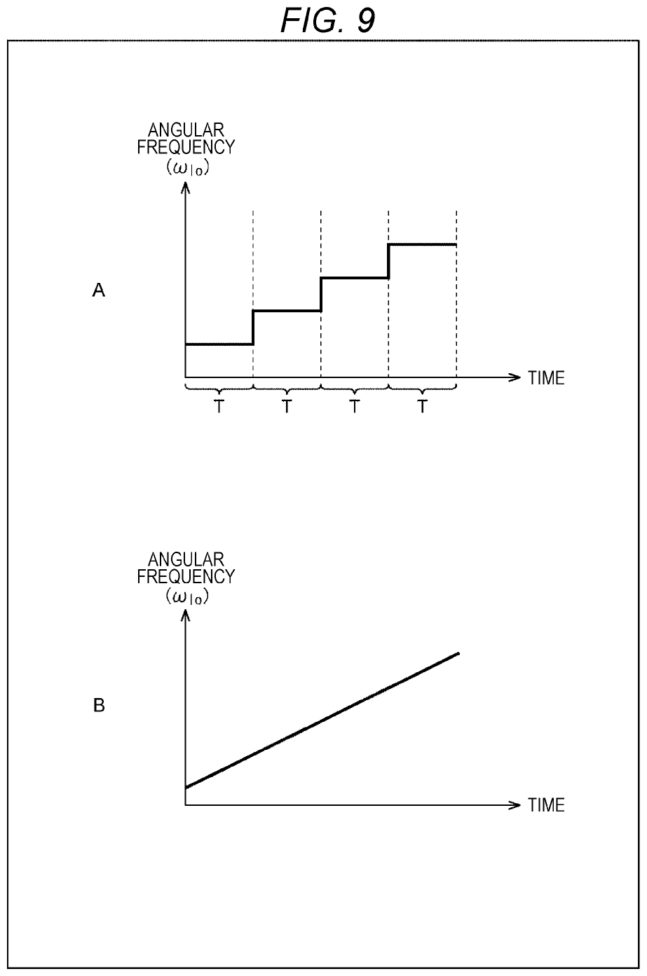

FIG. 9 is a diagram to describe exemplary sweep of an angular frequency ωₗₒ of a periodic signal in the local oscillator 140.

As illustrated in A of FIG. 9, the local oscillator 140 of FIG. 8 can sweep the angular frequency ωₗₒ such that the angular frequency ωₗₒ is increased in every fixed time period T, for example.

Furthermore, as illustrated in B of FIG. 9, the local oscillator 140 of FIG. 8 can sweep the angular frequency ωₗₒ such that the angular frequency ωₗₒ is continuously increased, for example.

Note that, in A and B of FIG. 9, the angular frequency ωₗₒ is swept so as to be increased with passage of time, but the angular frequency ωₗₒ can be swept so as to be decreased with passage of time.

Furthermore, the angular frequency ωₗₒ can be swept in every fixed time period T until certain time, and the angular frequency ωₗₒ can be continuously swept after the certain time.

In the extraction unit 115 illustrated in FIG. 8, the local oscillator 140 sequentially (in time series) generates N periodic signals having the different angular frequencies ω_{lo #n}=ωₗₒ₁, ωₗₒ₂, . . . , ω_{loN} by sweeping the angular frequency ωₗₒ. Furthermore, the mixing unit 121 sequentially mixes a reflection signal with each of the N periodic signals having the different angular frequencies ω_{lo #n}=ωₗₒ₁, ωₗₒ₂, . . . , ω_{loN}, and the LPF 123 sequentially extracts N extraction signals by sequentially filtering the respective N mixed signals acquired by the mixing.

Then, in the arithmetic operation unit 116 of FIG. 8, calculation of the pieces of power P(ωₗₒ₁), P(ωₗₒ₂), . . . , P(ω_{loN}) of the N extraction signals, multiplication of each angular frequency ω_{lo #n} by the power P(ω_{lo #n}) of each of the N extraction signals, and addition of N multiplication values ω_{lo #n}P (ω_{lo #n}) acquired by the multiplication are sequentially performed to obtain a blood flow velocity.

As described above, in the case where the extraction unit 115 and the arithmetic operation unit 116 include the one path as illustrated in FIG. 8, the blood flow velocity similar to that in the case of FIG. 7 can be obtained by sequentially performing the arithmetic operation performed in each of the paths in the case of including the N paths in FIG. 7. Then, in the case of configuring the extraction unit 115 and the arithmetic operation unit 116 like FIG. 8, it is not necessary to increase the number of paths even though the number N of the angular frequencies ω_{lo #n} is increased, and therefore, the circuit scale of the measurement device 100 can be prevented from being increased in proportion to the number N of angular frequencies ω_{lo #n}.

<Fourth Exemplary Configuration of Extraction Unit 115 and Arithmetic Operation Unit 116>

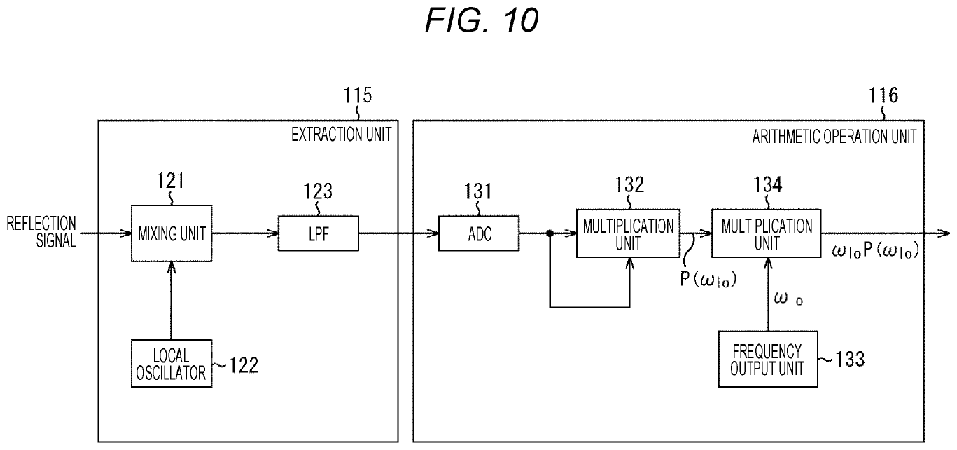

FIG. 10 is a block diagram illustrating a fourth exemplary configuration of the extraction unit 115 and the arithmetic operation unit 116.

In FIG. 10, the extraction unit 115 and the arithmetic operation unit 116 include only one path.

That is, the extraction unit 115 includes the mixing unit 121, the local oscillator 122, and the LPF 123. The arithmetic operation unit 116 includes the ADC 131, the multiplication unit 132, the frequency output unit 133, and the multiplication unit 134.

A reflection signal is supplied from the TIA 113 to the mixing unit 121, and a periodic signal having a certain angular frequency ωₗₒ is also supplied thereto from the local oscillator 122. The mixing unit 121 multiplies (mixes) the reflection signal supplied from the TIA 113 with the periodic signal of the certain angular frequency ωₗₒ supplied from the local oscillator 122 and supplies, to the LPF 123, a mixed signal acquired by the multiplication.

The local oscillator 122 generates the periodic signal having the certain angular frequency ωₗₒ and supplies the periodic signal to the mixing unit 121.

The LPF 123 filters the mixed signal supplied from the mixing unit 121 and supplies, to the ADC 131, an extraction signal (frequency component of a low angular frequency of the mixed signal) acquired by the filtering.

The ADC 131 performs AD conversion for the extraction signal supplied from the LPF 123 and supplies the converted extraction signal to the multiplication unit 132.

The multiplication unit 132 squares the extraction signal supplied from the ADC 131 to obtain power P(ωₗₒ) of the extraction signal, and supplies the power P(ωₗₒ) to the multiplication unit 134.

The frequency output unit 133 supplies, to the multiplication unit 134, the angular frequency ωₗₒ of the periodic signal used to obtain the extraction signal for which the power P(ωₗₒ) has been obtained by the multiplication unit 132.

The multiplication unit 134 multiplies the power P(ωₗₒ) of the extraction signal supplied from the multiplication unit 132 by the angular frequency ωₗₒ supplied from the frequency output unit 133 and outputs, as a blood flow velocity, a multiplication value ωₗₒP(ωₗₒ) acquired by the multiplication.

In the extraction unit 115 and the arithmetic operation unit 116 of FIG. 10, the blood flow velocity is obtained by using the power P(ωₗₒ) of the extraction signal obtained relative to the one certain angular frequency ωₗₒ, but the power P(ωₗₒ) of the extraction signal includes power in a frequency band having a width same as a passband width of the LPF 123 (LPF 123ₙ) according to the description in FIG. 3, and therefore, the accuracy of the blood flow velocity can be more improved than in the technology disclosed in Patent Document 1 in which a blood flow velocity is obtained focusing only on a specific frequency.

Furthermore, since the adding unit 135 is unnecessary in the arithmetic operation unit 116 illustrated in FIG. 10, a cost for the measurement device 100 can be more reduced than in the case where the arithmetic operation unit 116 is provided with the adding unit 135 as illustrated in FIGS. 4, 7, and 8.

Note that the present technology is applicable to measurement of a velocity of a fluid flowing inside a subject other than a human body, in addition to a blood flow velocity inside the human body.

Furthermore, the processing is performed for an analog signal in the extraction unit 115 in each of FIGS. 2, 7, 8, and 10, but the processing may be performed for a digital signal.

<Computer to which Present Technology is Applied>

Next, the series of processing of the extraction unit 115 and the arithmetic operation unit 116 described above can be performed by hardware or software. In the case of performing the series of processing by the software, a program constituting the software is installed in a computer.

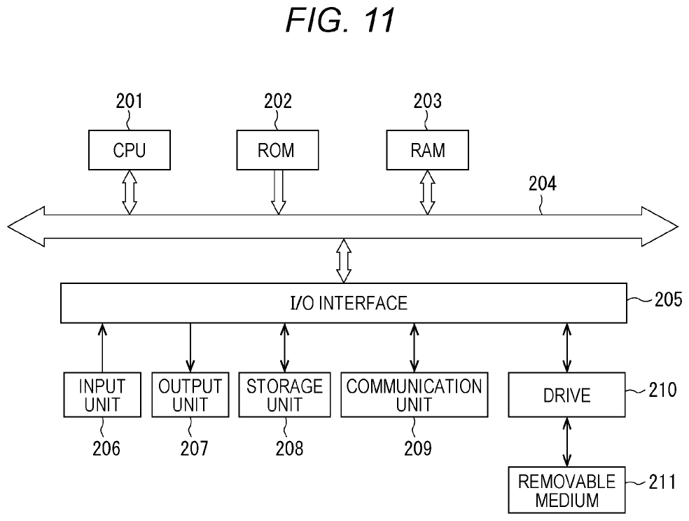

Considering such a case, FIG. 11 illustrates an exemplary configuration of an embodiment of the computer in which the program that executes the above-described series of processing is installed.

In FIG. 11, a central processing unit (CPU) 201 executes various kinds of processing in accordance with a program stored in a read only memory (ROM) 202 or a program loaded from a storage unit 208 to a random access memory (RAM) 203. The RAM 203 also stores data and the like required when the CPU 201 executes the various kinds of processing as appropriate.

The CPU 201, the ROM 202, and the RAM 203 are mutually connected via a bus 204. An I/O interface 205 is also connected to the bus 204.

An input unit 206 including a keyboard, a mouse, and the like; a display including a liquid crystal display (LCD) and the like; an output unit 207 including a speaker and the like; a storage unit 208 including a hard disk and the like; and a communication unit 209 including a modem, a terminal adapter, and the like are connected to the I/O interface 205. The communication unit 209 performs communication processing via, for example, a network such as the Internet.

A drive 210 is also connected to the I/O interface 205 as necessary and a removable medium 211 such as a magnetic disk, an optical disc, a magneto-optical disk, or a semiconductor memory is attached as appropriate, and a computer program read therefrom is installed in the storage unit 208 as necessary.

Note that the program executed by the computer may be a program by which the processing is performed in time series in accordance with the order described in the present specification, or may be a program by which the processing is performed in parallel or at necessary timing such as when the program is called.

The embodiment of the present technology is not limited to the above-described embodiment, and various kinds of modifications can be made within a range not departing from the gist of the present technology.

Note that the effects recited in the present specification are merely examples and not limited thereto, and effects other than those recited in the present specification may also be provided.

<Others>

The present technology can also adopt following configurations.

(1)

A signal processing device including:

a mixing unit that mixes a periodical periodic signal with a reflection signal corresponding to reflection light reflected at a subject; and

a low pass filter (LPF) that filters a mixed signal acquired by mixing the periodic signal with the reflection signal.

(2)

The signal processing device recited in (1), further including

a multiplication unit that multiplies power of an output signal of the LPF by an angular frequency of the periodic signal.

(3)

The signal processing device recited in (1), further including:

a plurality of the mixing units each of which mixes the reflection signal with each of a plurality of the periodic signals having different angular frequencies; and

a plurality of the LPFs each of which filters each of a plurality of mixed signals acquired by mixing the reflection signal with each of a plurality of the periodic signals.

(4)

The signal processing device recited in (3), further including:

a plurality of multiplication units each of which multiplies each power of an output signal of each of the plurality of the LPFs by an angular frequency of the periodic signal used to obtain the output signal; and

an adding unit that adds a plurality of multiplication values obtained by multiplying each power of an output signal of each of a plurality of the LPFs by the angular frequency of the periodic signal used to obtain the output signal.

(5)

The signal processing device recited in (1), further including:

a single unit of the mixing unit that sequentially multiplies the reflection signal by each of a plurality of the periodic signals having different angular frequencies; and

a single unit of the LPF that sequentially filters a plurality of mixed signals acquired by mixing the reflection signal with each of a plurality of the periodic signals.

(6)

The signal processing device recited in (5), further including:

a single multiplication unit that sequentially multiplies, relative to each of a plurality of the periodic signals, each power of an output signal sequentially output from the LPF by an angular frequency of the periodic signal used to obtain the output signal; and

an adding unit that sequentially adds a plurality of multiplication values which are sequentially output from the multiplication unit and acquired by multiplying each power of the output signal by the angular frequency of the periodic signal.

(7)

The signal processing device recited in any one of (1) to (6), further including

a light-receiving portion that receives the reflection light and outputs the reflection signal corresponding to the reflection light.

(8)

The signal processing device recited in any one of (1) to (7), further including

a light-emitting portion that irradiates the subject with light.

(9)

The signal processing device recited in (8), in which

the light-emitting portion emits light that is at least partially coherent.

(10)

The signal processing device recited in any one of (1) to (9), in which

the subject includes a human body.

(11)

A signal processing method including:

mixing a periodical periodic signal with a reflection signal corresponding to reflection light reflected at a subject; and

filtering, by a low pass filter (LPF), a mixed signal acquired by mixing the reflection signal with the periodic signal.

(12)

A program that causes a computer to function as:

a mixing unit that mixes a periodical periodic signal with a reflection signal corresponding to reflection light reflected at a subject; and

a low pass filter (LPF) that filters a mixed signal acquired by mixing the reflection signal with the periodic signal.

(13)

A measurement device including:

a light-emitting portion that irradiates a subject with light;

a light-receiving portion that receives reflection light of the light reflected at the subject and outputs a reflection signal corresponding to the reflection light;

a mixing unit that mixes the reflection signal with a periodical periodic signal;

a low pass filter (LPF) that filters a mixed signal acquired by mixing the reflection signal with the periodic signal; and

a multiplication unit that multiplies power of an output signal of the LPF by an angular frequency of the periodic signal.

### REFERENCE SIGNS LIST

100 Measurement device
111 Light-emitting portion
112 Light-receiving portion
113 TIA
114 Signal processing unit
115 Extraction unit
116 Arithmetic operation unit
121 Mixing unit
122 Local oscillator
123 LPF
131 ADC
132 Multiplication unit
133 Frequency output unit
134 Multiplication unit
135 Adding unit
140 Local oscillator
201 CPU
202 ROM
203 RAM
204 Bus
205 I/O interface
206 Input unit
207 Output unit
208 Storage unit
209 Communication unit
210 Drive
211 Removable disk

1. A signal processing device comprising:
a mixing unit configured to mix a periodical periodic signal with a reflection signal corresponding to reflection light reflected at a subject; and
a low pass filter (LPF) configured to filter a mixed signal acquired by mixing the periodic signal with the reflection signal.

2. The signal processing device according to claim 1, further comprising
a multiplication unit configured to multiply power of an output signal of the LPF by an angular frequency of the periodic signal.

3. The signal processing device according to claim 1, further comprising:
a plurality of the mixing units each configured to mix the reflection signal with each of a plurality of the periodic signals having different angular frequencies; and
a plurality of the LPFs each configured to filter each of a plurality of mixed signals acquired by mixing the reflection signal with each of a plurality of the periodic signals.

4. The signal processing device according to claim 3, further comprising:
a plurality of multiplication units each configured to multiply each power of an output signal of each of a plurality of the LPFs by an angular frequency of the periodic signal used to obtain the output signal; and
an adding unit configured to add a plurality of multiplication values obtained by multiplying each power of an output signal of each of a plurality of the LPFs by the angular frequency of the periodic signal used to obtain the output signal.

5. The signal processing device according to claim 1, further comprising:
a single unit of the mixing unit configured to sequentially multiply the reflection signal by each of a plurality of the periodic signals having different angular frequencies; and
a single unit of the LPF configured to sequentially filter a plurality of mixed signals acquired by mixing the reflection signal with each of a plurality of the periodic signals.

6. The signal processing device according to claim 5, further comprising:
a single multiplication unit configured to sequentially multiply, relative to each of a plurality of the periodic signals, each power of an output signal sequentially output from the LPF by an angular frequency of the periodic signal used to obtain the output signal; and
an adding unit configured to sequentially add a plurality of multiplication values which are sequentially output from the multiplication unit and acquired by multiplying each power of the output signal by the angular frequency of the periodic signal.

7. The signal processing device according to claim 1, further comprising
a light-receiving portion configured to receive the reflection light and output the reflection signal corresponding to the reflection light.

8. The signal processing device according to claim 1, further comprising
a light-emitting portion configured to irradiate the subject with light.

9. The signal processing device according to claim 8, wherein
the light-emitting portion emits light that is at least partially coherent.

10. The signal processing device according to claim 1, wherein
the subject includes a human body.

11. A signal processing method comprising:
mixing a periodical periodic signal with a reflection signal corresponding to reflection light reflected at a subject; and
filtering, by a low pass filter (LPF), a mixed signal acquired by mixing the reflection signal with the periodic signal.

12. A program that causes a computer to function as:
a mixing unit configured to mix a periodical periodic signal with a reflection signal corresponding to reflection light reflected at a subject; and
a low pass filter (LPF) configured to filter a mixed signal acquired by mixing the reflection signal with the periodic signal.

13. A measurement device comprising:
a light-emitting portion configured to irradiate a subject with light;
a light-receiving portion configured to receive reflection light of the light reflected at the subject and outputs a reflection signal corresponding to the reflection light;
a mixing unit configured to mix the reflection signal with a periodical periodic signal;
a low pass filter (LPF) configured to filter a mixed signal acquired by mixing the reflection signal with the periodic signal; and
a multiplication unit configured to multiply power of an output signal of the LPF by an angular frequency of the periodic signal.
